(19) United States
(12) Patent Application Publication
(10) Pub. No.: US 2024/0341575 A1
(43) Pub. Date: Oct. 17, 2024

(54) Intelligent in vivo Capsule Device with Electrodes

(71) Applicant: CAPSOVISION, Inc., Saratoga, CA (US)

(72) Inventor: Kang-Huai Wang, Saratoga, CA (US)

(21) Appl. No.: 18/629,921

(22) Filed: Apr. 8, 2024

Related U.S. Application Data
(60) Provisional application No. 63/459,207, filed on Apr. 13, 2023.
(60) Provisional application No. 63/460,307, filed on Apr. 18, 2023.

(51) Int. Cl. A61B 1/00; A61B 1/04; A61B 1/06; A61B 1/31

(52) U.S. Cl. CPC A61B 1/00147; A61B 1/000094; A61B 1/000095; A61B 1/00016; A61B 1/0002; A61B 1/00032; A61B 1/00105; A61B 1/00108; A61B 1/00131; A61B 1/041; A61B 1/042; A61B 1/0638; A61B 1/0655; A61B 1/0684; A61B 1/31

(57) ABSTRACT

A capsule camera device to adaptively use multiple spectrum light sources based on the image characteristics. The capsule camera device comprises at least two light sources to illuminate lumen walls of a GI tract, wherein said at least two light sources are capable of generating at least two spectrums; an image sensor to capture images from a scene illuminated by the light sources; an image processing module to determine image characteristics in one or more captured images; a battery to provide power to the capsule camera device; a control module coupled to said at least two light sources to cause said at least two light sources to generate one or more target spectrums depending on the image characteristics in said one or more captured images; and a housing to enclose said at least two light sources, the image sensor, the battery and the control module in a sealed environment.

### CROSS REFERENCE TO RELATED APPLICATIONS

The present invention is a non-Provisional Application of and claims priority to U.S. Provisional Patent Application No. 63/459,207, filed on Apr. 13, 2023 and U.S. Provisional Patent Application No. 63/460,307, filed on Apr. 18, 2023. The U.S. Provisional Patent Applications are hereby incorporated by reference in their entireties.

### FIELD OF THE INVENTION

The present invention relates to in vivo capsule device. In particular, the present invention relates to intelligent capsule with electrodes for movement control and/or at least two light sources capable of generating at least two spectrums.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Because of the difficulty traversing a convoluted passage, endoscopes cannot reach the majority of the small intestine and special techniques and precautions, that add cost, are required to reach the entirety of the colon. Capsule endoscope is an alternative in vivo image sensor that addresses many of these problems. A camera is housed in a swallowable capsule, along with a radio transmitter for transmitting data, primarily comprising images recorded by the digital camera, to a base-station receiver or transceiver and data recorder outside the body. Another autonomous capsule camera system with on-board data storage was disclosed in the U.S. patent application Ser. No. 11/533,304, filed on Sep. 19, 2006.

A typical procedure of imaging the gastrointestinal track based on a wireless capsule camera consists of an inpatient visit in the morning during which a clinician attaches the base station apparatus to the patient and the patient swallows the capsule. The system records images beginning just prior to swallowing and records images of the gastrointestinal (GI) tract until its battery becomes fully discharged. For the system based on an autonomous capsule camera, the images captured will be stored in on-board memory and there is no need to attach a base station to the patient. After the capsule camera is swallowed, peristalsis propels the capsule through the GI tract. The rate of passage depends on the degree of motility. Usually, the small intestine is traversed in 4 to 8 hours. It takes longer to travel through the entire GI tracts. In order to reduce the procedure time for examining the colon, medication is needed to increase the transit speed. Some patients have known slow transit speed and in general the transit time is even long while a patient is sleeping. It is desirable that a means for reducing the procedure time by applying an electrical stimulus to the capsule in vivo. While the capsule camera usually moves in the forward direction (i.e., toward the rectum), it may undergo a retrograde motion occasionally, particularly in the colon. Furthermore, the capsule camera may stay at one place for certain periods of time. Beside the capsule camera, there are other in vivo capsule devices used for various diagnosis and treatment purposes. Other capsule devices also encounter the same issue as the capsule camera. It is desirable to provide an apparatus and method to influence the movement of the capsule device in the GI tract. The capability to influence the movement of the capsule device may offer benefits to broad applications associated with a capsule device.

In U.S. patent Ser. No. 10/531,786, granted on Jan. 14, 2020, a Capsule Device with Electrodes is disclosed. In an embodiment according to the invention, the capsule incorporates an electrical terminal, where the electrical terminal is electrically connected to a circuit inside the capsule housing. The electrical terminal is also called an electrode. FIG. 1A illustrates an example of a capsule device 110 incorporating an embodiment of the present invention. The capsule device usually has an elongated shape having two ends in the longitudinal direction. In FIG. 1A, the electrical terminal or electrode is in the form of a stud 120, where the end of the stud facing toward outside of the capsule has a surface that is flush with the capsule housing, as shown in FIG. 1A. However, the stud need not be exactly flush to the housing outer surface as long as their recess or protrusion is small and the outer surface of the capsule, including the studs, is smooth. The capsule device shown in FIG. 1A corresponds to a capsule camera. Nevertheless, an embodiment according to the present invention may be incorporated in other capsule devices as well. In the example of FIG. 1A, the capsule device also includes archival memory 140, processor 150, camera 160, and batteries 170. The archival memory is used to store captured images. However, the archival memory may also be used to store other sensory data (such as temperature and PH values). If a wireless capsule camera is used, a wireless transmitter or a wireless transceiver (transmitter and receiver) is used. For the wireless capsule camera, the archival memory may not be needed or may be reduced. The camera typically comprises an image sensor (such as a CMOS or CCD image sensor), an optical system (not shown in FIG. 1A) to project an object in the field of view onto the image sensor, and image processing and compression module. The processor 150 may be used to support the image processing and compression functions and the compression functions may comprise motion-compensated video coding. Furthermore, the processor 150 may also be used to support other functions such as light control for LED light (not shown in FIG. 1A). While a processor module 150 is shown in FIG. 1A, the processor module may also be implemented using multiple processors and also may be physically separated into multiple modules.

While FIG. 1A illustrates an example of a single electrode incorporated in a capsule device to practice the present invention, a capsule device may also incorporate more than one electrode in the capsule device to practice the present invention. For example, FIG. 1B illustrates an example of a capsule device with two electrodes (121 and 122) at one end of the capsule device. Furthermore, a capsule device may also incorporate one or more electrodes on both ends. For example, FIG. 1C illustrates an exemplary capsule incorporating an electrode on each end of the capsule (123 and 123a) and FIG. 1D illustrates an exemplary capsule incorporating two electrodes on each end of the capsule (124, 125, 124a and 125a).

In one implementation, multiple electrodes may be configured around the longitudinal axis on one end of the capsule symmetrically, such as in a circular arrangement. The plane associated with the circle is preferred to be perpendicular to the longitudinal axis of the capsule to increase the chance that the electrodes will touch the mucosal surface. For example, FIG. 2A illustrates a bottom view of a capsule device incorporating 8 electrodes (220) at one end of the capsule camera, wherein the electrodes are configured in a circle and the plane of the circle is perpendicular to the longitudinal axis of the capsule device. While the circle is used as an example of electrode configuration to increase the chance that one or more electrodes will touch the mucosal surface, other configurations that dispose the electrodes around the perimeter of one end of the capsule device will serve the same purpose. In another implementation, an electrode may comprise an area of conductive surface 230 which is disposed on the surface of one end of the capsule device as shown in FIG. 2B. Each electrode also comprises a stud portion 240 that is used as feed-through portion to provide support for the conductive surface portion as well as to provide conductive connection from the conductive surface to other components inside the housing 110. The conductive surface (230) shown in FIG. 2B has a larger cross section to improve the chance of making contact with the mucosal surface. Nevertheless, the conductive surface 230 portion of the stud may be formed by extending the feed-through portion through the capsule housing and having the exposed end of the feed-through portion flush with the housing surface substantially.

In yet another implementation, an electrode is implemented with an area covered by traces of conductive surface material. These conductive traces are connected to other components or contact points inside the capsule via a conductive part such as a metal stud or a metal wire. The electrode may be connected to an internal signal or stimulus sources via the conductive part. In one implementation, feed-through is used to connect the internal circuit to the external terminal or electrode. The implementation of feed-through needs to maintain the hermetic seal of the capsule. In one example, adhesive is applied and cured to provide the seal.

The electrodes can be configured for multiple functions. For example, the electrode may be coupled to the storage device within the capsule to download data after the capsule is excreted from the human body. Furthermore, the electrode may also be used to exert electrical stimulus in vivo to stimulate the intestine muscle contraction to change motor activity and to cause the capsule to move faster or slower, or change direction of the movement. Therefore, the electrodes may be configured to provide the function of in vivo data transmission using the human body as an electrical media, where an electrical signal may be exerted on the electrodes to send signal to an external device coupled to the human body. The electrodes may also be configured to provide sensing function such as measuring electrochemical parameters within the GI track.

The electrodes can be coupled to a processing unit to provide the signal required to drive the electrodes or to process the signal sensed by the electrodes. The processing unit may be implemented in a state machine and a circuit module comprising logic circuits and/or controller. Furthermore, the electrodes may be coupled to a switching circuitry, where the switching circuitry may configure the electrodes to perform a desired function from a set of functions. For example, the electrodes may be configured to perform movement adjustment function when the capsule is inside the GI track. While the capsule device is inside the human body, the electrodes may be configured, by time sharing with the movement adjustment function, to collect electrochemical data in the GI tract. Alternatively, multiple sets of electrodes can be used for concurrent movement adjustment and electrochemical data. collection. Furthermore, the electrodes are configured to perform data download function after the capsule device exits from the human body. A system incorporating an embodiment of the present invention may comprise a switch module that selectively connects the electrodes to a respective module to perform the selected function. The modules performing various functions may be implemented on separate modules with respective processor and respective analog driving circuitry. Some of the separate modules may share some common components. Furthermore, the modules may also be implemented as a single integrated module. In one embodiment a programmable processor could perform different functions with different embedded firmware codes.

FIG. 3 illustrates a simplified example of three-function electrode for movement adjustment, data collection and data download. During movement adjustment function, the required signal for the electrodes is generated by signal generation module 310. In order to effectively adjust the capsule movement, the signal delivered to the electrodes may have to be brought to a certain voltage level or current level. The signal driver 320 is used to form the desired signal level for the electrodes 340. Two electrodes are shown in the example. However, more or less electrodes may be used. The switch 330 is used to properly connect a corresponding module to the electrodes. The switch 330 may be controlled by a controller or a system processor which is not shown in FIG. 3. While single lines are connected between various modules and the electrodes, each single line may represent multiple wires as appropriate. The movement or pose adjustment can cause the capsule device to move faster or slower, or to change direction or orientation to achieve better image capture during the capture mode.

In the data collection mode, the electrodes are connected to the signal amplifier/processing module 370 to gather the electrochemical data and stored in archival memory 350. In the data download mode, the data stored in the archival memory 350 is read out into the interface circuit 360 and the module is coupled to the electrodes 340 so that an external device may access data stored inside the capsule device through the electrodes 340 without the need to physically open the capsule device. FIG. 3 is intended for an illustration of an embodiment of the present invention, a person skilled in the art may practice the present invention with other variations without departing from the spirit of the present invention. While a single archive memory is shown in FIG. 3, separate archive memories may also be used for sensory data collection and image capture. In one embodiment, structured light derived distance to mucosa combined with motion vector for more accurate calculation of capsule movement can be used.

The capsule device is typically in an elongated shape so that the capsule device will travel primarily along the longitudinal direction. However, it is uncertain regarding which end of the capsule will be facing the forward direction when moving along the GI tract. Therefore, when a stimulus signal is applied to the electrode for adjusting the capsule movement, the capsule device may be inadvertently moved in a wrong direction. An embodiment according to the present invention incorporates a set of electrodes on each end of the capsule device and a direction determination module to determine the direction of the capsule device in the GI tract. Upon the direction determined and the desired movement adjustment, the system embodying the current invention may configure the capsule device to apply correspond stimulus signals to the electrodes. For example, if a movement adjustment to cause the capsule device move forward is desirable, a stimulus signal can be applied to the electrodes at the end of the capsule device corresponding to the backward end. The signal from the electrodes will stimulate the mucosal surface and cause it to contract. The capsule will move in the forward direction due to the intestine contraction caused by the electrical stimulus. When a stimulus needs to be applied to one end of the electrodes, the electrodes at the other end can be tri-stated, kept open, or high input/output resistance. In one embodiment, another set of switch may switch stimulus to either end of capsule.

In one implementation, the capsule device incorporates LED lights on four sides or more to illuminate the intestine wall. Based on light intensities received from different sides of the capsule, it can determine which side of the capsule is closer to the intestine wall. Alternatively, pose position derivation can be used to determine which side of the capsule is closer to the intestine wall. Accordingly, the electrodes on one side may be applied stronger or weaker stimulus than the other side at the same end of the capsule to steer the capsule into a desirable pose position.

In one implementation, a capsule camera is used as the capsule device and the capsule camera direction inside the GI tract is determined according to images captured by the capsule camera. Furthermore, motion estimation can be used to determine motion information associated with the capsule, such as capsule direction, capsule displacement, capsule movement or any combination. The motion estimation can be derived based on a subset of the images, such as spatially and/or temporarily sub-sampled images, or cropped images. Global motion estimation can be used to determine the direction of movement. Since the capsule device moves primarily in a forward direction in the GI track, the global motion can discern the direction of capsule device movement. Motion information such as motion vectors of a plurality of blocks could be used to determine the direction of the global motion. The average of these motion vectors, the mean, or the majority of these motion vectors, the peak of the histogram of these motion vectors, the median of the motion vectors, etc., can be used to determine the direction of the capsule device inside the GI tract.

To increase the accuracy in determining the direction of the capsule device inside the GI tract, the global motion vectors derived from images captured at different times over a time period can be used jointly to determine the direction of the capsule device inside the GI tract. For example, the average, mean, the majority of sign, the histogram, of the global motion vectors corresponding to images captured at different times over a time period can be used jointly to determine the direction of the capsule device inside the GI tract. Sometimes the capsule device may travel sideway, i.e., the axis of the capsule device corresponding to elongated direction is perpendicular to the longitudinal direction of the GI tract. The benefit of direction determination based on multiple global motion vectors over a period of time becomes apparent in this case. In one embodiment according to the present invention, no stimulus is exerted on the electrodes when decision of capsule direction inside the GI track cannot be decided. The process of determining capsule direction inside the GI tract based on images captured can be made in vivo by the capsule device or externally by an external device such as a base station. For a wireless capsule camera without on-board storage for image data, the captured images are transmitted to the base station through the wireless link. In such case, the process of determining capsule motion information such as direction, displacement and speed inside the GI tract can be made at the base station. The decision on capsule direction inside the GI tract can be communicated back to the capsule through the wireless link. The capsule camera can apply the stimulus signal to the electrodes according to the information received from the base station.

An exemplary block diagram is shown in FIG. 4 to illustrate a capsule camera 400 and a base station 450 incorporating an embodiment of the above invention. The capsule camera 400 comprises camera 410, wireless link 420, processor/driver 430, electrodes 440 and antenna 442. The function of processor 430 can be shared with the processor inside camera 410 or wireless link 420. For example, process 430 may include motion estimation function which can be shared by motion estimation required by video compression in camera 410. Camera 410 captures images and the captured images are transmitted using wireless link block 420 and antenna 442. Base station 450 receives the captured images through antenna 452 and wireless link 460. The received images are processed by processor 470, where motion information such as the amount of capsule movement and direction with respect to the GI tract is determined. The information of the determined amount capsule movement and direction with respect to the GI tract is transmitted back to the capsule camera. The capsule camera can determine the needed stimulus signal according to the determined capsule movement and direction and the desired movement adjustment. Alternatively, processor block 470 at base station 450 may further determine a control signal based on the determined capsule movement and direction and the desired movement adjustment and transmit the control signal to the capsule camera through wireless link 460. FIG. 4 is intended for illustration of an embodiment of the present invention, a person skilled in the art may practice the present invention with other variations without departing from the spirit of the present invention.

In U.S. Patent Application Publication No. 2016/0249793A1, an invention was disclosed for the use of the special spectrum light source or a second light source targeted for a certain type of lesion, cancerous, pre-cancerous, or otherwise abnormal condition. The use of the special spectrum light source or a second light source is limited to the intended sections of the gastrointestinal (GI) tract. For example, a patient has certain symptoms indicative of a certain section with pathologies or a patient has familial inheritance of a certain disease that normally exists in a certain section of the GI tract. In another example, a patient has some particular habits such as smoke or volume alcohol consumption that may result in certain section in the GI tract in need of special attention. In yet another example, a patient may have a certain pre-exiting condition such as GERD (Gastroesophageal reflux disease), Helicobacter pylori or previous colon polyp removal, which makes this patient a higher risk of malignancy in the esophagus, stomach or colon respectively. In yet another example, a certain section of the GI tract has higher dangerous pathologies than the other sections, such as the colon has higher likelihood of cancer growth than the small bowel.

The multiple light sources capable of generating multiple spectrum characteristics (or simply called spectrums) can be configured to generate different spectrums. For example, for the first spectrum, the light is more prominent in the shorter wave range (e.g., with a peak around 415 nm) and for the first spectrum, the light is more prominent in the longer wave range (e.g., with a peak around 700 nm). If more light sources are used, it is possible to generate more different spectrum by selectively turning on/off some light sources and/or applying different driving voltages to the light sources.

Furthermore, the lesion in different sections of the GI tract may have different spectral response characteristics. Therefore, different light sources or different groups of light sources may be needed for each section. According to U.S. Patent Application Publication No. 2016/0249793A1, different light sources having different spectral contents are applied to different sections of the GI tract. This may result in images more clearly indicative of pathological significance and can also achieve substantial power saving compared to the case of using all light sources indiscriminately.

White light is usually used for standard endoscopy, where the light source has a light spectrum that covers substantially all visible lights. Therefore, white light endoscope can produce images similar to visual inspection of the mucosa with the naked eye. The LED light is typically used for the capsule application due to its compactness and low power consumption.

It is known that different anatomical structure and tissue characteristics may respond to specific light spectrum differently. For example, blood vessels absorb visible light with a principle peak around the 415 nm (i.e., blue light). This makes the blood vessels seen clearly under light with this short wavelength. Accordingly, narrow band imaging has been used for various endoscopic applications such as to detect the hyper-vascular locations that often characterize the malignancy or precursor of malignancy. It is also known that cancer lesion has higher concentration of certain substances that have different spectrum responses from the nearby tissues. Therefore, relevance between anatomical structure/tissue characteristics and their responses to specific light spectrum can be utilized to detect the existence of possible malignancy or precursor of malignancy.

In one embodiment of U.S. Patent Application Publication No. 2016/0249793A1, images for an intended area of the GI tract may be taken under more than one light source, where each light source has a certain spectral characteristics. For example, a certain cancer lesion has different spectral responses. The images with combined light sources may reveal all features that are visible under lights with respective spectral responses. In another example, a section of the GI tract with two types of lesions having different spectral responses may take advantage of light sources with different corresponding spectral characteristics to reveal possible lesion in the images. In this case, light sources with different corresponding spectral characteristics are applied to the section of the GI tract where the two types of lesion are prone to occur. In yet another example, different images may be taken with one or more white light sources, one or more first special light sources with first spectral characteristic, and one or more second special light sources with second spectral characteristic for that section. The white light sources, the first special light sources and the second special light sources are applied in turn to illuminate the lumen during image capture. For example, the white light sources may be applied to capture the first image; the first special light sources may be used next to capture the second image; and then the second special light sources may be used to capture the third image for a substantially same scene. While the different types of light sources may be used in turn, these light sources may also be applied in a combined fashion. For example, the white light sources and the first special light sources may be used at the same time to illuminate a particular section of the GI tract. The white light sources and the second special light sources may be used at the same time to illuminate another particular section of the GI tract.

In the present invention, an intelligent capsule device with electrodes, which can be operated intelligently based on features of captured images, is disclosed.

### SUMMARY OF THE PRESENT INVENTION

A capsule camera device to adaptively use multiple spectrum light sources based on the image characteristics in said one or more captured images. The capsule camera device comprises at least two light sources to illuminate lumen walls of a GI (gastrointestinal) tract, wherein said at least two light sources are capable of generating at least two spectrums; an image sensor to capture images from a scene illuminated by said at least two light sources; an image processing module to determine image characteristics in one or more captured images; a battery to provide power to the capsule camera device; a control module coupled to said at least two light sources to cause said at least two light sources to generate one or more target spectrums depending on the image characteristics in said one or more captured images; and a housing to enclose said at least two light sources, the image sensor, the battery and the control module in a sealed environment.

In one embodiment, the image characteristics in said one or more captured images comprise pathological significance in said one or more captured images. In one embodiment, when the image characteristics in said one or more captured images are detected, the control module is configured to cause said at least two light sources to switch to a different spectrum from an existing spectrum.

In one embodiment, said at least two spectrums comprise a white light. In one embodiment, the control module is configured to cause said at least two light sources to generate the white light for capturing a first image; to generate first different spectrum for capturing a second image; and to generate second different spectrum for capturing a third image. In one embodiment, said at least two spectrums comprise a visible light with a principle peak around 415 nm.

Another capsule camera device is also disclosed. The capsule camera device comprises a capsule housing; one or more electrodes disposed fixedly through the capsule housing, wherein said one or more electrodes apply electrical stimulus to living body tissue in a patient's gastrointestinal tract; a signal generation or driver unit to generate the electrical stimulus for said one or more electrodes; an image sensor to capture images from a scene illuminated by a light source; an image processing module to determine image characteristics in one or more captured images; a control module coupled to said one or more electrodes to provide one or more control signals to said one or more electrodes depending on the image characteristics in said one or more captured images; and wherein the signal generation or driver unit, the image sensor, the image processing module and the control module are enclosed in the capsule housing.

In one embodiment, the capsule camera device further comprised at least two light sources to illuminate lumen walls of a GI (gastrointestinal) tract, wherein said at least two light sources are capable of generating at least two spectrums. In one embodiment, the control module or a second control module, coupled to said at least two light sources, selects one or more selected light sources from said at least two light sources depending on the image characteristics in said one or more captured images. In one embodiment, the image characteristics in said one or more captured images comprise pathological significance in said one or more captured images.

Yet another capsule camera device is also disclosed. The capsule camera device comprises a light source to illuminate lumen walls of a GI (gastrointestinal) tract; an image sensor to capture one or more images from a scene illuminated by the light source; an image capture control coupled to the image sensor to control image resolution of images captured; an image processing module to determine image characteristics in one or more captured images; a battery to provide power to the capsule camera device; a control module coupled to the image capture control to change image resolution of subsequent captured images depending on the image characteristics in said one or more captured images; and a housing to enclose said the light source, the image sensor, the battery and the control module in a sealed environment.

In one embodiment, the image resolution of subsequent captured images is set to high resolution when pathological significance in said one or more images is detected.

### BRIEF DESCRIPTION OF THE DRAWINGS

FIG. 1A-FIG. 1D illustrate four examples of electrode arrangement for the capsule device incorporating an embodiment of the present invention.

FIG. 2A illustrates an example of multiple electrodes configured in a circle at one end of the capsule device in the longitudinal direction.

FIG. 2B illustrates an example of electrode structure having an area of conductive surface disposed on the surface of one end of the capsule device in the longitudinal direction.

FIG. 3 illustrates an exemplary block diagram of a capsule device incorporating multi-function electrodes according to an embodiment of the present invention.

FIG. 4 illustrates an exemplary block diagram of a wireless capsule camera system using a base station to detect the capsule motion information or direction inside the GI tract.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention. Reference throughout this specification to “one embodiment,” “an embodiment,” or similar language means that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention. The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

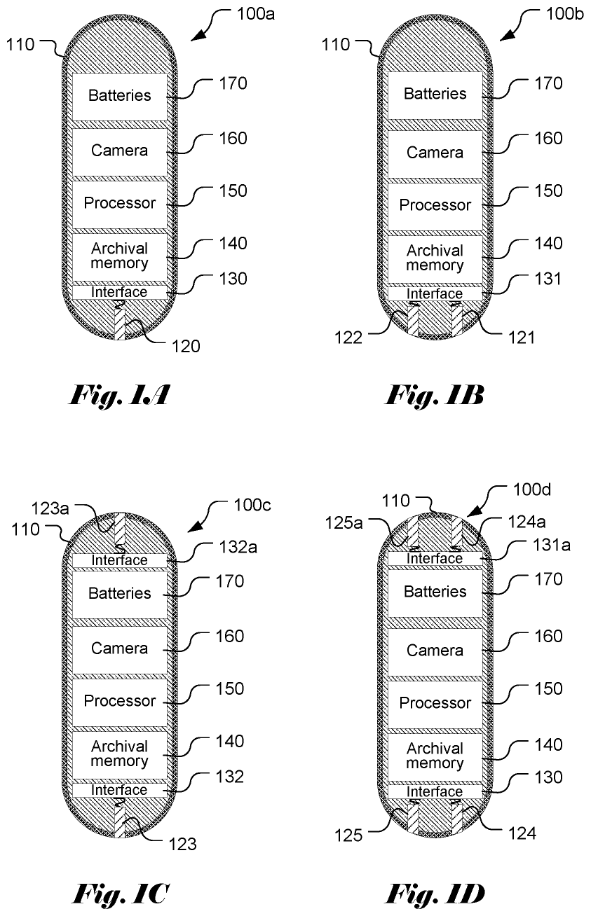

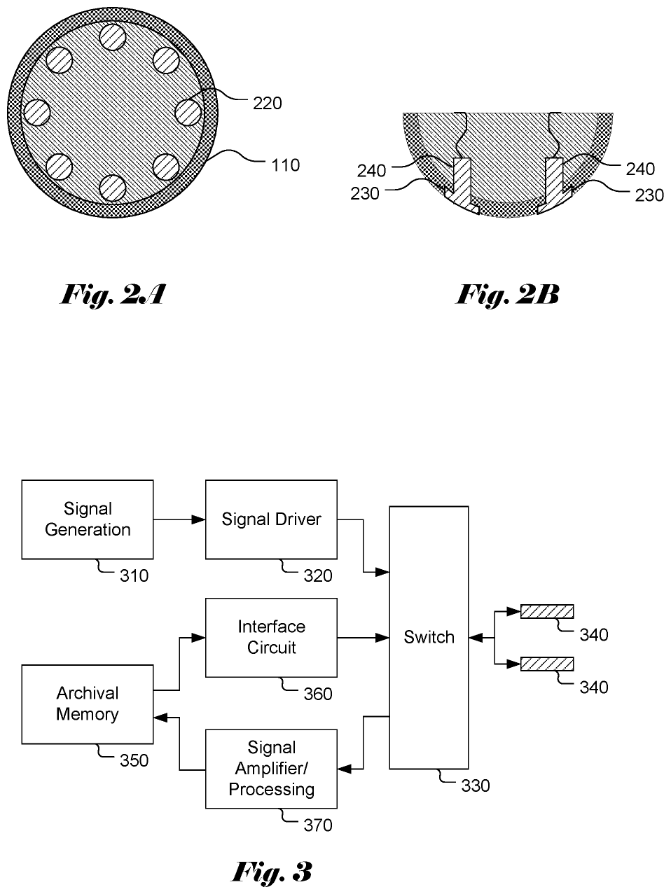

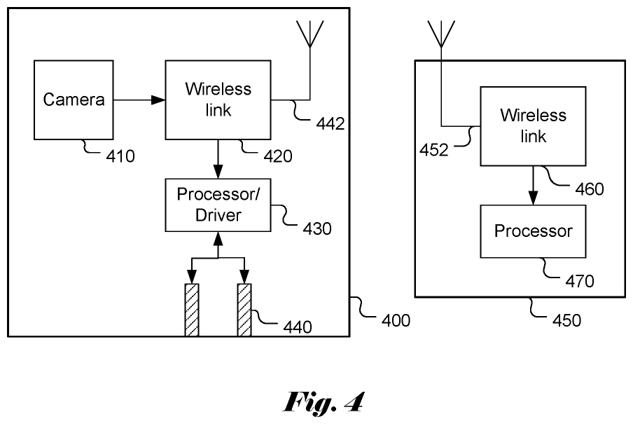

In FIG. 1 and FIG. 2, the electrodes are located at one end of the capsule along the longitudinal direction of the capsule. According to another embodiment, the electrodes are located slightly toward the center section of the capsule and arranged perpendicular to the longitudinal direction of the capsule.

In one embodiment, structured light derived distance to mucosa combined with motion vector for more accurate calculation of capsule movement can be used.

When the capsule device travels inside the GI tract, the differences in transit time through the intestine among people are widespread. On one hand, when the capsule device moves too slowly inside the GI tract, it may be desired to increase the transit speed in the forward direction. On the other hand, when the capsule device moves too fast inside the GI tract, it may be desired to decrease the transit speed in the forward direction. When stimulus is needed to adjust the capsule device movement, the capsule direction inside the GI tract has to be known before the stimulus signal is applied. The motion vector-based direction determination may be used to determine the capsule direction inside the GI tract. Sometimes different sections of GI tract of the same person may have different transit speeds and the stimulus is needed in some part of the intestine to increase or to decrease the speed. In some sections of the GI tract, it may be preferred to have more images taken per distance travelled. Therefore, the control for adjusting capsule device movement may be different for different sections of the GI tract. The control for adjusting the capsule device movement may include criteria regarding when to apply the stimulus and the strength or length of the stimulus signal. Since the control for adjusting capsule device movement may be dependent on particular sections of the GI tract, such as the colon section and the small bowel section, the system may need to discern the different sections of the GI track. In one embodiment of the present invention, the ratio of light intensity from the light sources versus the pixel values received by the image sensor is used to determine the different sections of the GI tract.

When the capsule device is a capsule camera, the images captured by the capsule camera can be used to discern the speed of the capsule device In this case, the image sensor inside the capsule device, which forms an integral part of a capsule camera, is used as the sensing device for determining the amount of capsule movement and direction inside the GI tract. As mentioned before, motion estimation may be used to determine the capsule movement between captured images. The distance travelled by the capsule camera can be divided by the corresponding time interval to determine the transit speed.

As disclosed in U.S. patent Ser. No. 10/531,786, the movement of an in vivo capsule device can be controlled by using electrodes and applying control signals to the electrodes. In order to properly control the movement of the capsule device, the strength, duration and frequency of the stimulus have to be determined on the fly. For different individuals, the required strength and frequency of the stimulus to cause desired capsule movement may be different. Therefore, the strength and frequency of the stimulus is determined adaptively based on the responses of individuals to the applied stimulus according to an embodiment of the present invention. For example, if a stimulus applied does not cause anticipated motor activity or transit speed change, then the control module may increase the intensity of each application of the stimulus or increase the duration of each application of the stimulus. Alternatively, the control module may increase the frequency of the applying the stimulus. For safety reason, there is an upper limit on the strength of the stimulus. In one embodiment adjusting duty cycle or pulse width modulation is used to control speed.

The bowel transit is episodic in nature and the need to apply the stimulus may be made on a longer period of time according to a predetermined schedule and/or location. For example, it may take about 4 hours for a capsule device to reach the cecum after the device is swallowed, or it may take the capsule device 10 hours to travel about 5 meters to reach the cecum. Accordingly, the capsule device may be configured to activate the movement adjust function upon a predetermined schedule or when the capsule device reached the intended location. The stimulus strength and duration will be adapted to the individual as well as the specific section of GI tract to achieve desired response of motor activity. In one embodiment the stimulus is applied simply after certain hours, for example 8 hours.

The process of determining the capsule direction inside the GI tract should be performed before the stimulus is applied to the electrodes. Consequently, there is a period of time that the capsule does not exert stimulus on the electrodes. This period of time for determining the capsule direction inside the GI tract is needed for the capsule device to gather multiple images and to make decision so as to be accurate and to avoid false determination causing the capsule to retrograde. Sometimes, the bowel activity is very low and this will make determination of the direction difficult. An embodiment according to the present invention may exert stimulus signals on both ends of the capsule (electrodes incorporated on both ends are assumed). The stimulus signals exerted on both ends of the capsule are substantially equal in intensity and duration in order to agitate the bowel momentarily. The stimulus signals can be applied to both ends substantially at the same time. The stimulus signals will trigger peristalsis to cause the capsule device to move by peristalsis. The direction detection can be performed and the determined direction can be used for capsule movement control by applying an appropriate stimulus to the electrodes at the correct end of the capsule. The series of process including initially applying the stimulus signal to cause the capsule device move due to agitated bowel, detecting the capsule direction and applying stimulus signal to cause a desire capsule device movement, is called stimulus decision mode. The process of capsule movement control, as described above, by exerting stimulus is always performed after the capsule direction is determined. However, the stimulus may also be applied while the decision is being made.

Due to the advancement in hardware technology, the size of capsule device may be further shrunk. When the small-size capsule device travels in a wider section of the GI tract, the capsule device may flip sometimes causing the forward end to become the rear end. Therefore, the decision regarding which end of the capsule is facing forward direction has to be made frequently. However, if the speed detection shows that the capsule moves at the desired speed during a period of time, then there is no need to perform direction detection and no stimulus is needed either.

For a very small capsule device, the capsule device tends to turn more easily. In order to detect the capsule device turn, pose estimation is utilized. The pose (i.e., the three dimensional position and orientation with respect to a coordinate system) of the capsule device can be estimated from images captured by the capsule device using pose estimation technique. Techniques for pose estimation are well known in the field of machine vision and the details are not repeated here. In an ideal case, the capsule longitudinal axis should be aligned with the axis perpendicular to the cross section of the GI tract and the capsule faces a forward direction. If the capsule tilts, there will be an angle between the two axes. The angle between the two axes can be determined from the pose of the capsule device. If the angle exceeds a threshold, it may be desirable to apply a stimulus to the capsule device to reduce the tilt so that the capsule device will not turn inadvertently.

For a wireless capsule device, the images can be transmitted to a base station so that the capsule pose information can be estimated at the base station. The estimated pose information can be transmitted to the capsule device so that the capsule device can adjust the speed and/or orientation according to the estimated pose information. Alternatively, the base station can determine the required control signal based on the estimated pose information and transmit the control information to the capsule device. If the capsule device uses on-board memory to store the captured images, the capsule device has to estimate the pose information using processor inside the capsule device.

The capsule as disclosed above can be configured for therapeutic applications, such as treatment of diseases (e.g. constipation or other diseases) by propelling the capsule to move and/or by stimulating the muscles. The electrodes of the capsule device can stimulate digestive nerves for improving mood or other conditions. Furthermore, the processor 150 as shown in FIGS. 1A-D, can incorporate Artificial Intelligence (AI) to cause the necessary stimulus data. The processor can also collect and analyze the data from human response, for example, by image data and the analyses of these data thereof, etc.

The capsule device with electrodes can be controlled to adjust the movement of the capsule device. The movement can be adapted to knowledge regarding the surrounding environments or the status of the capsule device. For example, at a certain section of the human GI tract, it may be desirable to slow down the movement of the capsule device so that more images can be taken. For example, it may be desirable to take more images at the descending colon. Therefore, control signals may be applied to the electrodes to slow down the movement of the capsule device. Furthermore, if some images with pathological importance are detected, it may be desirable to slow down or even cause the capsule device undergo a retrograde motion so that more images can be taken for the area with pathological significance. In other words, the capsule device may be moved intelligently with knowledge regarding the in vivo environment, such as the corresponding GI part associated with the location of the capsule device, or any location inside the GI tract with pathological significance. In one embodiment, the image quality can be detected, for example, by AI. If the quality of certain images is not of good quality, the controlled capsule movement may be desired to take one or more image of adequate quality.

Accordingly, the present invention discloses an intelligent capsule device, where the movement of the capsule device is controlled depending on the knowledge regarding the in vivo environment. In particular, the movement of the capsule device in controlled depending on detection of important pathology in the GI tract. There are various techniques to detect whether there is any pathological significance within one or more images. For example, in U.S. Pat. No. 10,068,334, issued on Sep. 4, 2018, a technique is disclosed to estimate the surface depth. Accordingly, pathological features such as subsurface tumors can be detected from the estimated depth map using this method. In one embodiment of the present invention, if such pathological features indicating any pathological significance is detected, the capsule device is controlled so that more images can be taken. For example, the capsule device can be controlled to slow down its movement speed or even to cause retrograde motion.

In another example, the pathological significance can be detected based on color profile of images. For example, if the color profile of an area in an image is highly concentrated in red, this may be an indication of bleeding or inflammation in the area. In the field of image processing, there are various techniques to detect or recognize certain features in the images. These techniques can be used to detect pathological significance in the images. In recent years, neural networks or artificial intelligence have been applied to medical imaging to help automatic detection of certain medical conditions or anomalies. These techniques can be used to detect any pathological significance in captured images.

In another application, the processor may be configured to make intelligent movement according to some data analysis or characteristics of the images. For example, the processor may analyze the capture images to determine if an image contains some important pathology. If so, the capsule could go backwards by electrode stimulation and take more pictures. Alternatively, the capsule device may capture pictures with a higher resolution. We might incorporate AI inside capsule to detect pathology, landmark or other conditions real time, or close to real time. To capture high resolution images, image sensors with a large number of pixels will be required, which implies smaller pixel size is used. Due to the energy required to process a large number of pixels, and the light energy to provide adequate illumination to smaller pixels, higher definition images in capsule endoscope may be taken only when the situation is warranted, such as pathological significance is detected. Otherwise images with standard resolution may be taken, such as 100K or 250K or 500K pixels per image. Let's use 250K as the standard resolution for example. The image sensor may be able to support 1M pixels. In a regular resolution mode, pixel binning can be used to combine the signals from neighboring 4 pixel of the same color, in analog domain or digital domain, to operate it at 250K resolution, while in high resolution mode all 1M pixel are read out. In one embodiment the lighting has to be stronger in the high resolution mode to keep signal out of each pixel adequate without compromising signal to noise ratio. The higher resolution images can be taken only at certain important landmarks or pathologies.

In yet another application, upon detection of any pathological significance or target characteristics in the images, the capsule device equipped with multiple-spectrum lighting sub-system can be controlled to illuminate the scene with a different light spectrum. The pathologies sometimes are more conspicuous under certain spectrum or a certain combination of spectrums. For example, when dysplasia or cancer happens, microvascular overgrowth may occur. Because the blood absorbs certain spectrum in certain visible light, so the light rich in that spectrum will make the pathology more obvious. There may be multiple spectrum bands that the blood absorbs more efficiently. The visible light rich in those spectrum bands will make the pathology more obvious. Similarly, certain molecules associated with a cancer may absorb certain spectrums more than other spectrums. Therefore, the light rich in those spectrums will make the pathology more visible. The motion estimation, with or without the assistance of information of distance to mucosa, will guide the controlled movement to the desired location, and the AI could recognize if the pathology is within the FOV.

Accordingly, in another embodiment of the present invention, the control of multiple-spectrum lighting subsystem is dependent on the detected characteristics of the images. For example, the detected characteristics of the images may correspond to pathological significance. For example, the capsule device may be operated with a white light originally. Upon the detection of any pathological significance in the images, images can be taken with one or more different spectrums. For example, upon the detection of any pathological significance in the image, the multiple-spectrum lighting subsystem can be switched to a first different spectrum to take one or more images; then the multiple-spectrum lighting subsystem can be switched to a second different spectrum to take one or more images again. Furthermore, the process can be continued to go through all available spectrums from the multiple-spectrum lighting subsystem. In another embodiment, depending on the detected pathological significance or the location of the capsule device within the GI tract, a selected set of spectrums among all available spectrums can be used for imaging the neighborhood where pathological significance is detected.

In yet another embodiment, if the capsule device with the multiple-spectrum lighting subsystem is also equipped with electrodes, the movement of the capsule device can be controlled along with multiple-spectrum lighting control. For example, when pathological significance is detected, the movement of the capsule device can be slowed down or made to undergo retrograde motion when images are taken with different spectrums. In this case, an area with pathological significance can be imaged using various spectrums. Therefore, images with one or more different spectrums may show enhanced pathological features, which is useful to help physician for diagnosis.

An embodiment of the present invention to perform the steps disclosed in this application can be based on an application specific integrated circuit (ASIC), a microcontroller, or a hardware-based processor. An embodiment of the present invention may also be program codes to be executed on a Digital Signal Processor (DSP) to perform the processing described herein. The invention may also involve a number of functions to be performed by a computer processor, a digital signal processor, a microprocessor, or field programmable gate array (FPGA). These processors can be configured to perform particular tasks according to the invention, by executing machine-readable software code or firmware code that defines the particular methods embodied by the invention. The software code or firmware code may be developed in different programming languages and different format or style. The software code may also be compiled for different target platform. However, different code formats, styles and languages of software code and other means of configuring code to perform the tasks in accordance with the invention will not depart from the spirit and scope of the invention.

The above description is presented to enable a person of ordinary skill in the art to practice the present invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the above detailed description, various specific details are illustrated in order to provide a thorough understanding of the present invention. Nevertheless, it will be understood by those skilled in the art that the present invention may be practiced.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A capsule camera device, comprising:
at least two light sources to illuminate lumen walls of a GI (gastrointestinal) tract, wherein said at least two light sources are capable of generating at least two spectrums;
an image sensor to capture images from a scene illuminated by said at least two light sources;
an image processing module to determine image characteristics in one or more captured images;
a battery to provide power to the capsule camera device;
a control module coupled to said at least two light sources to cause said at least two light sources to generate one or more target spectrums depending on the image characteristics in said one or more captured images; and
a housing to enclose said at least two light sources, the image sensor, the battery and the control module in a sealed environment.

2. The capsule camera device of claim 1, wherein the image characteristics in said one or more captured images comprise pathological significance in said one or more captured images.

3. The capsule camera device of claim 1, wherein when the image characteristics in said one or more captured images are detected, the control module is configured to cause said at least two light sources to switch to a different spectrum from an existing spectrum.

4. The capsule camera device of claim 1, wherein said at least two spectrums comprise a white light.

5. The capsule camera device of claim 4, wherein the control module is configured to cause said at least two light sources to generate the white light for capturing a first image; to generate first different spectrum for capturing a second image; and to generate second different spectrum for capturing a third image.

6. The capsule camera device of claim 1, wherein said at least two spectrums comprise a visible light with a principle peak around 415 nm.

7. A capsule camera device comprising:
a capsule housing;
one or more electrodes disposed fixedly through the capsule housing, wherein said one or more electrodes apply electrical stimulus to living body tissue in a patient's gastrointestinal tract;
a signal generation or driver unit to generate the electrical stimulus for said one or more electrodes;
an image sensor to capture images from a scene illuminated by a light source;
an image processing module to determine image characteristics in one or more captured images;
a control module coupled to said one or more electrodes to provide one or more control signals to said one or more electrodes depending on the image characteristics in said one or more captured images; and
wherein the signal generation or driver unit, the image sensor, the image processing module and the control module are enclosed in the capsule housing.

8. The capsule camera device of claim 7, further comprising at least two light sources to illuminate lumen walls of a GI (gastrointestinal) tract, wherein said at least two light sources are capable of generating at least two spectrums.

9. The capsule camera device of claim 8, wherein the control module or a second control module, coupled to said at least two light sources, selects one or more selected light sources from said at least two light sources depending on the image characteristics in said one or more captured images.

10. The capsule camera device of claim 9, wherein the image characteristics in said one or more captured images comprise pathological significance in said one or more captured images.

11. A capsule camera device, comprising:
a light source to illuminate lumen walls of a GI (gastrointestinal) tract;
an image sensor to capture one or more images from a scene illuminated by the light source;
an image capture control coupled to the image sensor to control image resolution of images captured;
an image processing module to determine image characteristics in one or more captured images;
a battery to provide power to the capsule camera device;
a control module coupled to the image capture control to change image resolution of subsequent captured images depending on the image characteristics in said one or more captured images; and
a housing to enclose said the light source, the image sensor, the battery and the control module in a sealed environment.

12. The capsule camera device of claim 11, wherein the image resolution of subsequent captured images is set to high resolution when pathological significance in said one or more images is detected.
